(19) United States
(12) Patent Application Publication
(10) Pub. No.: US 2025/0353010 A1
(43) Pub. Date: Nov. 20, 2025

(54) ALL-IN-ONE CARTRIDGE AND DIAGNOSTIC DEVICE FOR DIAGNOSING DISEASES

(71) Applicant: IGENTECH CO. LTD., Seoul (KR)

(72) Inventor: Kyungho KIM, Seoul (KR)

(21) Appl. No.: 18/866,534

(22) PCT Filed: Jun. 23, 2023
(86) PCT No.: PCT/KR2023/008749

§ 371 Date: Nov. 16, 2024

(30) Foreign Application Priority Data
Aug. 2, 2022 (KR) 10-2022-0095796

(51) Int. Cl. B01L 3/00

(52) U.S. Cl. CPC B01L 3/502761; B01L 3/502715; B01L 2200/04; B01L 2200/0668; B01L 2200/10; B01L 2200/16; B01L 2300/021; B01L 2300/044; B01L 2300/0663; B01L 2300/0672; B01L 2300/1805

(57) ABSTRACT

The all-in-one cartridge (100) for on-site diagnosis provided in the present disclosure relates to an integrated cartridge capable of sample solution injection and detection. The cartridge is equipped with a sample injection cap (13), a sample injection port (14), a tip (12), a 2D barcode (15), a sample chamber (22), a buffer chamber (23), a mixing chamber (25), a washing chamber (26), and a PCR chamber (28) for performing PCR. The cartridge (100) can be used for molecular diagnosis, and a diagnostic device therefor can be selectively used for molecular diagnosis, immunodiagnosis and rapid diagnosis.

### TECHNICAL FIELD

The present disclosure relates to a cartridge used in a device for diagnosing diseases by using biological materials such as proteins and DNA. The cartridge provided by the present disclosure is an all-in-one POC cartridge for on-site diagnosis by which the proteins or DNA can rapidly be diagnosed, and is a cartridge having an integrated structure by which a sample can be injected into the cartridge provided by the present disclosure and detected with the naked eye.

### BACKGROUND ART

Recently, the global spread of respiratory infectious viruses such as COVID-19 has become a major issue, and, in addition thereto, interest in a healthy living environment has grown. Accordingly, technologies for rapid on-site diagnosis of the presence or absence of diseases are expanding.

In particular, in the case of molecular diagnostics or immunodiagnostics characterized by the detection of proteins or DNA, as the automation of molecular tests and immunoassay techniques become complex, there is no product that provides adequate performance for clinical use within a near-patient testing facility. A typical molecular test involves various steps comprising lysis of cells, purification, and amplification for subsequent detection, in order to accurately inject a reagent, introduce a sample, and extract DNA or RNA. While there are central laboratory robotic platforms that automate such steps, central laboratories are unable to provide results within a required time frame on many tests that require a short turnaround time.

However, it is difficult to implement a system within a clinical setting that provides accurate and reliable results at a reasonable cost. Given the complex nature of various molecular testing techniques, if test parameters are not carefully controlled or environmental conditions are not ideal, significant errors in diagnostic results may easily occur.

In particular, in order to detect proteins or DNA, a diagnostic method comprising extracting the biomaterial, carrying out a gene amplification process while accommodating the biomaterial in a biochip made of materials such as glass, silicon, metal, and plastic, irradiating the biochip with a light source, and analyzing the amount of light (fluorescence) emitted from the biochip, is recently used. In the diagnostic method, the extracted biomaterial is injected into the biochip and loaded into a diagnostic device. Such a device used to extract a certain biomaterial to be diagnosed among the biomaterials extracted from the body of a living organism is called a cartridge.

In the past, there were few cases where extraction was performed while such a cartridge was accommodated in a diagnostic device. Generally, a device used to manufacture biochips was manufactured separately, and a biochip manufactured in the device was fed into a diagnostic device manufactured for diagnostic purposes. However, this method had many cases that the continuity between the manufacturing of the biochip and the diagnostic process was interrupted such that a lot of time was required for diagnosis, or the biomaterial leaked outside the biochip, contaminating the device, which caused significant errors in the diagnostic results.

### PRIOR ART CITATIONS

### Patent Citations

Korean Patent No. 10-1642434

### CONTENTS OF THE INVENTION

### Technical Problems

The objective of the present disclosure is to solve the problems of conventional molecular diagnosis that it takes a long time to perform various processes involving cell lysis, purification and subsequent amplification for detection in order to accurately inject a reagent, introduce a sample, and extract DNA or RNA; and the continuity of the manufacturing of a biochip that introduces a biomaterial and a diagnostic process is interrupted. The all-in-one field diagnosis cartridge provided by the present disclosure can solve the above-described problems by integrating the tip where a sample is loaded and detected in a diagnostic device after the sample is directly injected.

### Means of Solving Problem

The all-in-one cartridge for on-site diagnosis provided in the present disclosure is manufactured in a rectangular parallelepiped shape, has a sample inlet formed on one side of the cartridge, and comprises a sample chamber, a buffer chamber, a mixing chamber, a washing chamber, and a PCR chamber for storing a sample injected from the sample inlet, has an aluminum foil fused thereto for protecting the injected sample, and has a structure in which a flexible thin sealing film is assembled to an upper cover and a lower cover and may move left and right, and a tip assembled to the sealing film is formed in a structure in which the tip can move up and down and left and right on the upper part of the chamber.

In addition, the cartridge provided by the present disclosure can perform molecular diagnosis or immunodiagnosis depending on the target to be detected, by using a separate module, without the need for changing the cartridge and the diagnostic device that performs the diagnosis by inserting the cartridge.

### Effect of the Invention

By using the all-in-one cartridge for on-site diagnosis provided by the present disclosure, a diagnostic process can be performed quickly by detecting proteins or DNA, and the time required to detect the proteins or DNA can be significantly reduced, and the reliability of the diagnostic results can be improved by forming the injection and detection of the sample as an integrated structure. In addition, the cartridge provided by the present disclosure can perform molecular diagnosis or immunodiagnosis depending on the target to be detected, by using a separate module, without the need for changing the cartridge and the diagnostic device that performs the diagnosis by inserting the cartridge in the same diagnostic device.

### BRIEF DESCRIPTION OF THE DRAWINGS

FIG. 1 shows the overall shape of a cartridge provided in the present disclosure.

FIG. 2 shows various chambers provided inside a cartridge provided in the present disclosure.

FIG. 3A shows the process of sealing a PCR chamber when conducting PCR diagnosis by using a cartridge provided in the present disclosure.

FIG. 3B shows the state in which a PCR chamber is sealed when conducting PCR diagnosis by using a cartridge provided in the present disclosure.

FIG. 4 shows the front of a diagnostic device loaded with a cartridge provided in the present disclosure.

FIG. 5 shows the back of a diagnostic device loaded with a cartridge provided in the present disclosure.

### DETAILED DESCRIPTION OF THE EMBODIMENTS

Hereinafter, preferred embodiments of the present disclosure will be described in detail with reference to the attached drawings. Prior to this, terms or words used in the specification and claims of the present disclosure should not be interpreted as being limited to their conventional or dictionary meanings, but should be interpreted as meanings and concepts that conform to the technical idea of the present disclosure, based on the principle that an inventor can appropriately define the concept of a term in order to explain his or her own invention in the best way. Therefore, the embodiments described in the specification of the present disclosure and the configurations illustrated in the drawings are only the most preferred embodiments of the present disclosure and do not represent all of the technical idea of the present disclosure. Therefore, it should be understood that there may be various equivalents and modified examples that can replace them at the time of filing the present application.

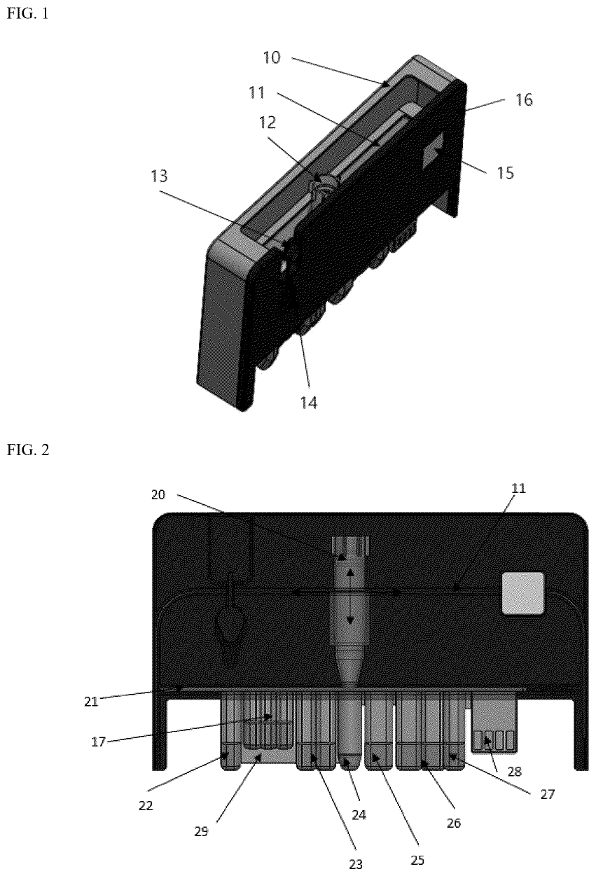

FIG. 1 is a perspective view of an all-in-one cartridge (100) provided by the present disclosure. As shown in FIG. 1, the cartridge (100) of the present disclosure is covered with an upper cover (10) and a lower cover (16), a sample injection cap (13) and a sample injection port (14) are formed on the outside of the cartridge (100), a tip (12) into which an injected sample is loaded is provided on the inside, and a 2D barcode (15) is installed on the lower surface of the cartridge (100).

A sample solution containing a target protein or DNA is injected into a sample injection port (14) by using a pipette and the like and, after the sample solution is injected, the sample injection cap (13) is closed to seal the sample solution.

The 2D barcode (15) has a code set in advance to recognize whether it is a molecular diagnosis mode or an immune diagnosis mode, depending on whether the target to be diagnosed by using the cartridge (100) of the present disclosure is a protein or DNA.

According to the mode (module) set in the above 2D barcode (15), the diagnostic device (300) that diagnoses by using the cartridge (100) provided in the present disclosure performs an operation process suitable for the molecular diagnosis mode or the immunodiagnosis mode. By such settings, the cartridge (100) and the diagnostic device (300) provided in the present disclosure can perform various modules in one device.

FIG. 2 depicts various components constituting the cartridge (100) provided in the present disclosure.

According to FIG. 2, the lower part (17) of the cartridge (100) is provided with a sample chamber (22) for sealing the injected sample solution, a buffer chamber (23) containing a solution for lysis and binding for loading the sample solution injected by the tip (12) to perform molecular diagnosis, a mixing chamber (25), a washing chamber (26), a buffer chamber (27) for containing a solution for elution, and a PCR chamber (28) for performing PCR. In addition, an extraction and freeze-drying chamber (29) for extracting and freeze-drying a target from the sample solution may be further provided.

In addition, the upper part of the tip (12) for loading the sample solution is provided with a tip filter (20) for preventing foreign substances from penetrating the sample solution and preventing contamination from the outside.

In addition, in order to prevent evaporation and movement of the solution due to distribution when performing a diagnosis by using the cartridge (100) provided in the present disclosure, the upper surface of the lower part (17) of the cartridge is sealed with aluminum foil (21).

In addition, in order to prevent the sample solution inside the chambers from evaporating to the top of the cartridge (100) or being contaminated to the outside when performing molecular diagnosis, a flexible and thin sealing film (11) which has a structure that allows the tip (12) to move up and down, simultaneously has a structure that can move left and right, and is assembled with the upper cover (10) and the lower cover (16) is provided.

As shown in FIG. 2, when performing molecular diagnosis or immunodiagnosis by using the cartridge (100), the tip (12) moves up and down by the syringe module in the sample chamber (22) to load the sample solution. In addition, in order to move the loaded sample solution to the buffer chamber (23), mixing chamber (25), and PCR chamber (28), the tip (12) is designed to be moved left and right by a motor (not shown). The tip (12) moves left and right by the sealing film (11) that divides the upper space of the cartridge (100) into upper and lower parts. To this end, the tip (12) and the sealing film (11) are assembled to move as one unit.

In addition, the PCR chamber (28) is formed as a thin film type heat-melting chamber structure, and the thin film type heat-melting chamber structure is a structure for increasing the ramping speed (heating, cooling) of the heat amplification cycle process of PCR. By using the cartridge (100) of the present disclosure, the PCR amplification speed is improved, thereby improving the diagnosis speed.

The PCR chamber (28) can be formed with a four-chamber structure, in which case sixteen fluorescences may be implemented by using four chambers and four fluorescent dyes.

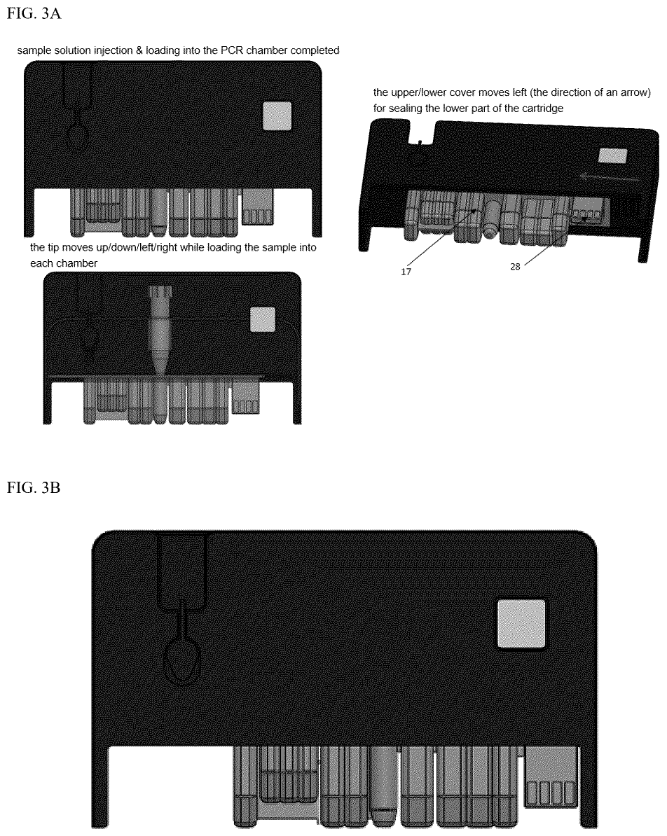

FIG. 3A and FIG. 3B show a sealing process to prevent the cartridge (100) from being contaminated by thermal amplification of PCR in the present disclosure, when performing molecular diagnosis through a PCR process by using the provided cartridge (100).

FIG. 3A shows a state in which, after a sample solution is injected, the tip (12) loads the sample solution to perform sample pretreatment, and the sample solution is loaded into the PCR chamber (28), and the upper cover (10) and the lower cover (16) show a preliminary step for sealing the lower part (17) of the cartridge. FIG. 3B shows the steps in which a sample solution is loaded into the PCR chamber (28), the upper cover (10) and the lower cover (16) move left and right while the lower part (17) of the cartridge is fixed to prevent evaporation or contamination of the sample solution during the PCR amplification process, and the inlet of each PCR chamber (28) is sealed to completely seal the cartridge (100).

In addition, the cartridge (100) provided in the present disclosure can perform molecular diagnosis, immunodiagnosis, and rapid diagnosis (rapid kit), depending on the biological material to be detected, and each of the diagnostic processes can be performed differently depending on the code set in the 2D barcode (15) provided on the lower surface of the cartridge (100).

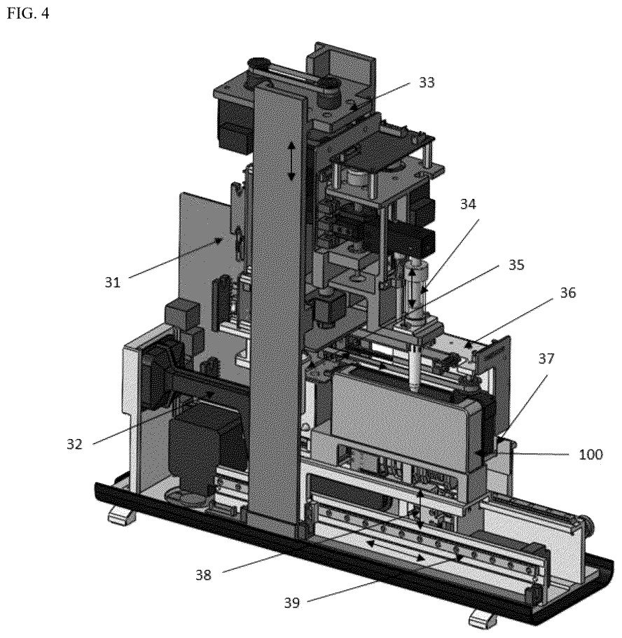

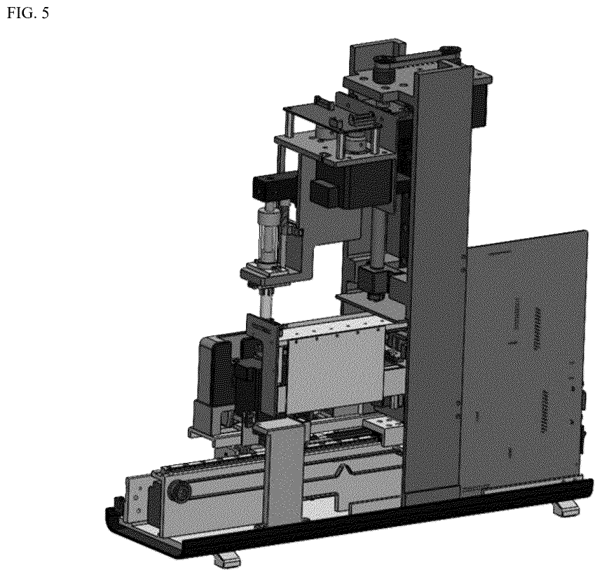

FIGS. 4 and 5 show a diagnostic device (300) that performs molecular diagnosis, immunodiagnosis, and rapid diagnosis by loading a cartridge (100) provided in the present disclosure.

According to FIG. 4, the diagnostic device (300) is formed by comprising a cartridge loading unit (39) for loading the cartridge (100), a cartridge heating unit (38) for heating the cartridge, a magnetic separation unit (37) for separating a sample solution, a multiplex detection unit (36) for molecular diagnosis, a detection unit (35) for immunodiagnosis, a syringe unit (34) for inhaling and discharging a predetermined amount of solution, a tip unit (33) for driving the syringe unit (34) up and down, a duct and fan (32) for heat circulation, and a main circuit unit (31) for performing the processes.

When performing a diagnostic process by using the cartridge (100) provided in the present disclosure, codes indicating molecular diagnosis, immunodiagnosis and rapid diagnosis are set in advance in the 2D barcode (15) of the cartridge (100), and, when the cartridge (100) is loaded into the diagnostic device (300), the diagnostic device (300) automatically recognizes the code set in the 2D barcode (15) and performs molecular diagnosis, immune diagnosis, rapid diagnosis, etc., respectively.

It is common knowledge to those skilled in the art that, although the present disclosure has been described with reference to the above drawings, the technical idea of the present disclosure is not limited to the above-mentioned drawings, and can be extended to all configurations that comprise the technical features of the present disclosure.

BRIEF DESCRIPTION OF THE REFERENCE NUMERALS

| | |
|---|---|
| 100 Cartridge | |
| 10 Upper cover | 11 Sealing film |
| 12 Tip | 13 Sample injection cap |
| 14 Sample injection port | 15 2D barcode |
| 16 Lower cover | 17 Lower part of the cartridge |
| 20 Tip filter | 21 Aluminum foil |
| 22 Sample chamber | 23 Buffer chamber (lysis and |
| | binding) |
| 24 Tip chamber | 25 Mixing chamber |
| 26 Washing chamber | 27 Buffer chamber (elution) |
| 28 PCR chamber | 29 Extraction and freeze-drying |
| | chamber |
| 300 Diagnostic device | |
| 31 Main circuit unit | 32 Duct and fan for heat circulation |
| 33 Tip unit | 34 Syringe unit |
| 35 Detection unit for | 36 Detection unit for molecular |
| immunodiagnosis | diagnosis |
| 37 Magnetic separation unit | 38 Cartridge heating unit |
| 39 Cartridge loading unit | |

1. An all-in-one cartridge (100), characterized in that:
the cartridge (100) is covered with a top cover (10) and a lower cover (16),
a sample injection cap (13) and a sample injection port (14) are formed on the outside of the cartridge (100),
a tip (12) into which the injected sample solution is loaded is provided on the inside of the cartridge (100),
a 2D barcode (15) is installed on the bottom of the cartridge (100), and
the bottom of the cartridge (100) is provided with a sample chamber (22) for sealing the injected sample solution, a buffer chamber (23) containing a solution for lysis and binding for loading the sample solution injected by the tip (12) to perform molecular diagnosis, a mixing chamber (25), a washing chamber (26), a buffer chamber (27) for containing a solution for elution, and a PCR chamber (28) for performing PCR, and is sealed with aluminum foil (21).

2. The all-in-one cartridge (100) according to claim 1, characterized in that:
the lower part of the cartridge (100) is further provided with an extraction freeze-drying chamber (29) for extracting and freeze-drying the target from the sample solution.

3. The all-in-one cartridge (100) according to claim 1, characterized in that:
the upper part of the tip (12) is further provided with a tip filter (20) to prevent foreign substances from penetrating into the sample solution.

4. The all-in-one cartridge (100) according to claim 1, characterized in that:
the PCR chamber (28) of the cartridge is formed with a heat-melting chamber structure.

5. The all-in-one cartridge (100) according to claim 1, characterized in that:
the PCR chamber (28) is formed with four heat-melting chamber structures.

6. The all-in-one cartridge (100) according to claim 1, characterized in that:
the cartridge (100) further comprises a sealing film (11) that divides the upper space into upper and lower parts,
the tip (12) and the sealing film (11) are assembled integrally, and
the cartridge (100) is sealed such that the tip (12) may move left and right, and the tip (12) may move up and down.

7. The all-in-one cartridge (100) according to claim 1, characterized in that:
the upper part of the PCR chamber (28) is sealed such that the lower part of the cartridge (17) is fixed, and the upper cover (10) and the lower cover (16) move left and right, in order to prevent the cartridge (100) from being contaminated by the thermal amplification of PCR in the course of performing a diagnosis through a PCR process by using the cartridge (100).

8. The all-in-one cartridge (100) according to claim 1, characterized in that:
one of the diagnostic processes, molecular diagnosis, immunodiagnosis or rapid diagnosis, is performed through a 2D barcode (15) provided in the cartridge (100).

9. A diagnostic device (300), characterized in that:
the diagnostic device has a diagnostic detection unit (35, 36) capable of loading a cartridge (100) of claim 1 to perform a diagnosis.

10. The diagnostic device according to claim 9, characterized in that:
the diagnostic device (300) performs one of molecular diagnoses, immunodiagnosis or rapid diagnosis, through a 2D barcode (15) provided in the cartridge (100).
